(19) United States
(12) Patent Application Publication
(10) Pub. No.: US 2018/0271154 A1
(43) Pub. Date: Sep. 27, 2018

(54) AEROSOL PROVISION SYSTEM WITH REMOTE AIR INLET

(71) Applicant: Nicoventures Holdings Limited, London (GB)

(72) Inventors: David LEADLEY, London (GB); Richard HEPWORTH, London (GB); My-Linh TRAN, London (GB)

(21) Appl. No.: 15/762,013

(22) PCT Filed: Sep. 12, 2016
(86) PCT No.: PCT/GB2016/052808

§ 371 Date: Mar. 21, 2018

(30) Foreign Application Priority Data
Sep. 22, 2015 (GB) 1516792.7

(51) Int. Cl. A24F 47/00; A61M 15/06; A61M 11/04

(52) U.S. Cl. CPC A24F 47/008; A61M 15/06; A61M 2205/8206; A61M 2205/3653; A61M 11/042

(57) ABSTRACT

A vapor provision system includes an aerosol delivery section configured to generate aerosol from liquid in a reservoir, an airflow path through the aerosol delivery section extending from an air inlet to a mouthpiece, a battery section configured to join to the aerosol delivery section and house a battery to provide electrical power to one or more components in the aerosol delivery section, the battery section arranged laterally to at least a portion to the aerosol delivery section with respect to a direction of airflow through the mouthpiece, and an interface region in which a surface of the aerosol delivery section faces a surface of the battery section when the sections are joined, the air inlet being located on the aerosol delivery section in the interface region so as to take in air that has been channeled over part of the battery section.

### PRIORITY CLAIM

The present application is a National Phase entry of PCT Application No. PCT/GB2016/052808, filed Sep. 12, 2016, which claims priority from GB Patent Application No. 1516792.7, filed Sep. 22, 2015, each of which is hereby fully incorporated herein by reference.

### TECHNICAL FIELD

The present disclosure relates to an aerosol or vapor provision system with an air inlet.

### BACKGROUND

Aerosol provision systems such as e-cigarettes generally contain a reservoir of a source liquid containing a formulation, typically including nicotine, from which an aerosol is generated, such as through vaporization or other means. Thus an aerosol source for an aerosol provision system may comprise a heating element coupled to a portion of the source liquid from the reservoir. When the heating element is activated it causes vaporization of a small amount of the source liquid, which is thus converted to an aerosol for inhalation by the user. More particularly, such devices are usually provided with one or more air inlet holes which may or may not be located away from a mouthpiece of the system. When a user sucks on the mouthpiece, air is drawn through the inlet holes and past the aerosol source. There is an air flow path connecting the inlet holes to the aerosol source and on to an opening in the mouthpiece so that air drawn past the aerosol source continues along the flow path to the mouthpiece opening, carrying some of the aerosol from the aerosol source with it. The aerosol-carrying air exits the aerosol provision system through the mouthpiece opening for inhalation by the user.

Some aerosol provision systems are configured in two sections. An aerosol provision section houses the reservoir of source liquid and one or more heating elements, and has the airflow path defined therethrough from the inlet hole(s) to the mouthpiece. A battery section houses a battery (which may be replicable or rechargable) for providing electrical power to the heating element. An electrical connection is provided between the two sections. The sections can be separable from one another, in which case there is also a mechanical connection between the sections; this typically also makes the electrical connection.

The two sections can be arranged linearly so that the battery section is connected at the opposite end of the aerosol provision section to the mouthpiece. This gives a generally elongate device in which the battery is aligned substantially along the direction of airflow in the flow path, and when the mouthpiece points upwards, as it does in use, the battery section is underneath the aerosol provision section. The air inlet can be located in a side wall of the aerosol delivery section just below the base of the reservoir, that is, the part of the reservoir remote from the mouthpiece. This gives a short and simple air flow path to the heating element (which is generally in or near the reservoir). However, there is a risk that the user will cover this air inlet with his hand or fingers when using the device. As well as inhibiting or reducing aerosol delivery, this can be unsafe in devices in which the heating element is switch-operated under control of the user. A lack of airflow can then lead to overheating.

### SUMMARY

According to a first aspect of certain embodiments described herein, there is provided a vapor provision system comprising: an aerosol delivery section configured to generate aerosol from liquid in a reservoir; an airflow path through the aerosol delivery section extending from an air inlet to a mouthpiece; a battery section configured to join to the aerosol delivery section and house a battery to provide electrical power to one or more components in the aerosol delivery section, the battery section arranged laterally to at least a portion to the aerosol delivery section with respect to a direction of airflow through the mouthpiece; an interface region in which a surface of the aerosol delivery section faces a surface of the battery section when the sections are joined; wherein the air inlet is located on the aerosol delivery section in the interface region so as to take in air that has been channeled over part of the battery section.

The battery section may be arranged laterally to the aerosol delivery section with respect to a direction of airflow through the mouthpiece.

The battery section may have a connecting portion that extends laterally to receive a base part of the aerosol delivery section, the air inlet being located in a base wall of the aerosol delivery section that faces the connecting portion when the sections are joined.

The vapor provision system may then further comprise co-operating screw threads or engaging elements on the connecting portion and the aerosol delivery section base part by which the sections can be joined, the screw threads or engaging elements being shaped such that when they are fully engaged, air can flow over at least part of the screw threads or engaging elements to be taken in by the air inlet.

Alternatively, a surface of the connecting portion that faces the base part of the aerosol delivery section has formed therein at least one recess such that when the sections are joined a cavity is formed in the interface region with at least one external opening to air at an edge of the interface region, the air inlet being in airflow communication with the cavity.

The at least one recess may comprise at least one groove in the said surface of the connecting portion, the or each groove extending radially with respect to a central axis of the aerosol delivery section when joined to the battery section to an end that forms one of the said at least one external openings.

The base part of the aerosol provision section and the said surface of the connecting portion may be shaped to form a central cavity in the interface region with which each groove is in airflow communication at an end opposite to the external opening end, the air inlet being in airflow communication with the central cavity.

The central cavity may house an electrical connection between the battery section and the aerosol delivery section.

In an alternative embodiment the aerosol delivery section and the battery section may be externally shaped such that when they are joined a cavity is formed in the interface region with at least one external opening to air at an edge of the interface region, the air inlet being in airflow communication with the cavity.

Said surface of the battery section in the interface region may be a side wall of the battery section and the said surface of the aerosol delivery section in the interface region may be a side wall of the aerosol delivery section.

The cavity is at least partially formed by at least one recess in the said side wall of the battery section having at least one end that forms one of said external openings to air. For example, each recess may be a groove extending across the said side wall of the battery section from a first end to a second end, each of the first and second ends forming one of said external openings to air. Alternatively, each recess may have an end forming one of said external openings to air, said end located at an edge of the interface proximate the mouthpiece.

The cavity may have at least two external openings to air.

The vapor provision system may further comprise an adjustable element configured to enable an effective size of the air inlet to the altered, so as to vary the level of airflow along the airflow path.

According to a second aspect of certain embodiments described herein, there is provided an aerosol delivery section for a vapor provision system which is configured to generate aerosol from liquid in a reservoir when joined to a battery section housing a battery, the aerosol delivery section comprising: an air inlet; an airflow path through the aerosol delivery section from the air inlet to a mouthpiece; and an interface region comprising a surface of the aerosol delivery section configured to face a surface of a battery section when the aerosol delivery section is joined to said battery section; wherein the air inlet is located in the interface region so as to take in air that has been channeled over part of the battery section when the sections are joined. The air inlet may be concealed from a user when the aerosol delivery section is joined to a battery section. In some examples, the air inlet may be located in a base wall or a side wall of the aerosol delivery section.

According to a third aspect of certain embodiments described herein, there is provided a battery section for a vapor provision system which is configured to house a battery to provide electrical power to an aerosol delivery section when the battery section is joined to an aerosol delivery section, the battery section comprising an interface region comprising a surface of the battery section configured to face a surface of an aerosol delivery section when the battery section is joined to said aerosol delivery section; and at least one recess formed in the interface region positioned for alignment with an air inlet in said aerosol delivery section, the recess having at least one end at an edge of the interface region to define an external opening to air. The battery section may be configured such that an aerosol delivery section may be joined to it in an arrangement in which the battery section is arranged laterally to at least a portion to the aerosol delivery section with respect to a direction of airflow through a mouthpiece of the aerosol delivery section. The battery section may have a base portion that extends laterally to which an aerosol delivery section may be joined.

These and further aspects of certain embodiments are set out in the appended independent and dependent claims. It will be appreciated that features of the dependent claims may be combined with each other and features of the independent claims in combinations other than those explicitly set out in the claims. Furthermore, the approach described herein is not restricted to specific embodiments such as set out below, but includes and contemplates any appropriate combinations of features presented herein.

### BRIEF DESCRIPTION OF THE DRAWINGS

Various embodiments will now be described in detail by way of example only with reference to the accompanying drawings in which:

FIG. 1 shows a schematic side view representation of an electronic cigarette to which embodiments of the disclosure are applicable.

FIG. 1A shows an enlarged view of a lower part of the electronic cigarette of FIG. 1.

FIGS. 2A and 2B show simplified side and top views of an electronic cigarette with regions of particular interest to the present disclosure highlighted.

FIG. 3 shows a schematic cross-sectional view of an example aerosol delivery section of an electronic cigarette in accordance with an embodiment of the disclosure.

FIG. 4 shows a schematic cross-sectional view of a further example aerosol delivery section coupled to a battery section.

FIGS. 5A, 5B and 5C show an enlarged partial cross-sectional view, a plan view and a side view of parts of an electronic cigarette in accordance with an embodiment.

FIGS. 6A and 6B show an enlarged partial cross-sectional view and a plan view of parts of an electronic cigarette in accordance with another embodiment.

FIGS. 7A and 7B show an enlarged partial cross-sectional view and a plan view of parts of an electronic cigarette in accordance with a further embodiment.

FIG. 8 shows a perspective view of a battery section of an electronic cigarette in accordance with an embodiment.

FIGS. 9A and 9B show side views of a battery section and an aerosol delivery section in accordance with a further embodiment.

FIGS. 10, 11 and 12 show side views of three example battery sections configured in accordance with further embodiments.

FIGS. 13A and 13B show a side view and a top view of a battery section in accordance with an alternative embodiment.

FIG. 14 shows a side view of battery section in accordance with a different embodiment.

FIG. 15 shows a cross-sectional side view of an electronic cigarette in accordance with a yet further embodiment.

FIG. 16 shows a schematic side view of an aerosol delivery section according to various embodiments.

### DETAILED DESCRIPTION

Aspects and features of certain examples and embodiments are discussed/described herein. Some aspects and features of certain examples and embodiments may be implemented conventionally and these are not discussed/described in detail in the interests of brevity. It will thus be appreciated that aspects and features of apparatus and methods discussed herein which are not described in detail may be implemented in accordance with any conventional techniques for implementing such aspects and features.

As described above, the present disclosure relates to aerosol provision systems, such as e-cigarettes. Throughout the following description the terms “e-cigarette” or “electronic cigarette” may sometimes be used; however, it will be appreciated these terms may be used interchangeably with aerosol (vapor) provision system.

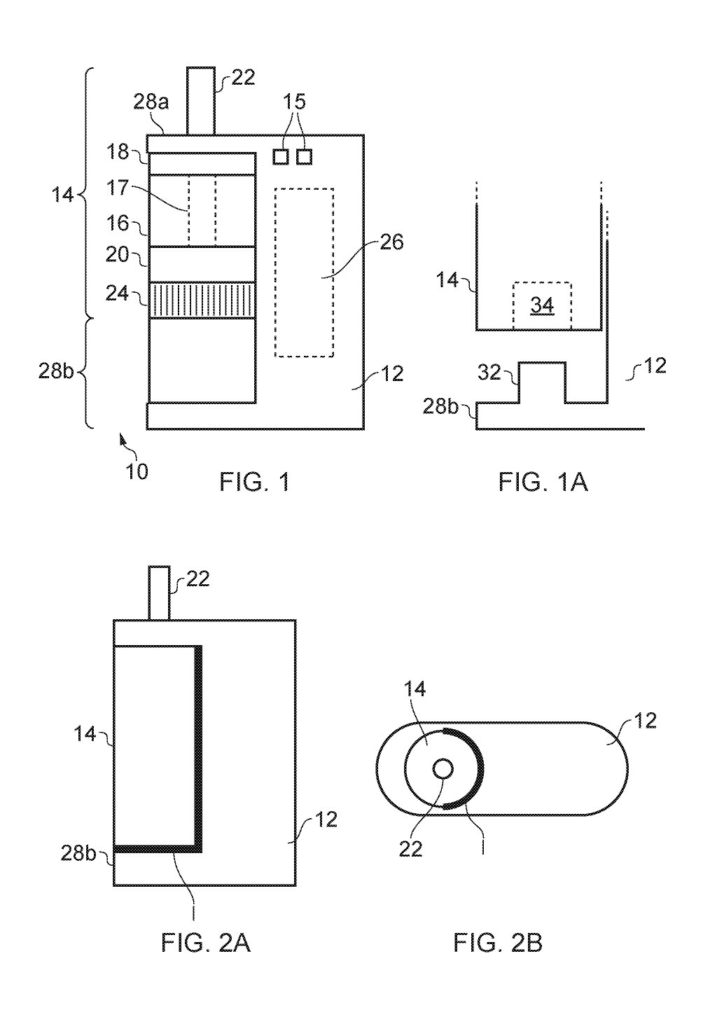

Directional terms in the present application, such as upper, lower, top, bottom, side and the like, are not to be considered limiting and are used for convenience and brevity, and consistency with the Figures. The terms apply to the typical orientation of an e-cigarette in use, when the mouthpiece points upward (such as in FIG. 1), and should be interpreted accordingly if considering a different orientation of an e-cigarette.

FIG. 1 is a schematic diagram of an example aerosol/vapor provision system such as an e-cigarette 10 to which some embodiments are applicable. The e-cigarette 10 is a modular device comprising a battery section 12 and an aerosol delivery section 14 which are mechanically and electrically connected together. The battery section 12 houses a battery 26 and has one or more buttons or switches 15 for a user to operate to deliver electrical power to one or more components in the aerosol delivery section 14. The battery section 12, in this example, has two supporting sections 28a, 28b which extend laterally to receive and support the aerosol delivery section 14. In particular, the lower supporting section 28b is a connecting portion which makes the necessary electrical connections to the battery 26, and also provides a mechanical connection which may be, for example, a screw thread connection between cooperating screw threads on the connection portion 28b and the base of the aerosol delivery section 14, or a friction “push fit” style of connection. The aerosol delivery section 14 is cylindrical in this example, and at its base has a grip portion 24 provided with vertical grooves or other surface features to facilitate grip, so that a user can hold this part and rotate the aerosol delivery section 14 to disengage the screw threads, or pull to overcome the friction fit. Alternatively the two sections may be shaped to slot or fit together, with a latch, clip, hinged portion or other retaining element provided to secure the two section together in the joined configuration. Other joining arrangements may also be used. Once the relevant parts are disengaged or separated, the aerosol delivery section 14 can be lifted free from the battery section 12.

FIG. 1A shows a simplified schematic partial side view of the lower part of the system 10 according to an example. The aerosol delivery section 14 is shown separated from the battery section 12. The battery section has an upwardly protruding connector 32 extending from the upper face of the connecting portion 28b, where the upper face is a wall or surface that faces the aerosol delivery section when the two sections are joined. The connector 32 is an electrode in a suitable housing or cover, by which an electrical connection can be made between the battery in the battery section 12 and components such as a heating element in the aerosol delivery section 14. The aerosol delivery section has a corresponding socket 34 formed inwardly on its lower face or wall which fits over the connector 32 and houses one or more electrical contacts which are brought into contact with the electrode to form the electrical connection when the sections are joined together.

The aerosol delivery section 14 comprises the aforementioned grip section 24 at its base, above which is a tank base section 20. A tank or reservoir 16 extends upwardly from this base section 20 and is formed by transparent walls so that a user can conveniently observe liquid solution contained in the tank 16. The walls need not be transparent, however. A tube or pipe 17 runs centrally up through the tank 16; this defines part of the airflow path that runs through the aerosol delivery section 14. Disposed within the pipe 17 are one or more wicks mounted within or around one or more heating elements that might be in the form of coils (not shown). The wicks absorb liquid from the tank, the heating coils are heated when electrical current is supplied to them from the battery 26, and the liquid in the wicks is vaporized, and carried away on air flowing through the pipe 17. A lid 18 is provided to close the upper end of the tank 16. The lid 18 can be removed to allow the tank 16 to be refilled when the aerosol delivery section 14 is disconnected from the battery section 12. The airflow path passes through the lid 18 to a mouthpiece 22 through which a user can inhale to generate the required airflow along the airflow path. The mouthpiece, also known as a “drip tip”, may or may not be removable and/or replaceable.

The opposite end of the air flow path within the aerosol delivery section 14 to the mouthpiece 22 is defined by at least one air inlet (not shown in FIG. 1) in the outer surface of the aerosol delivery section 14 which connects to the pipe 17. When a user inhales through the mouthpiece 22, air is taken in through the air inlet to flow along the air flow path.

Locating the air inlet in an area such as the exposed side of the tank base section 20 provides a conveniently short path to the heating coils and wick. However, an inlet in this location is vulnerable to being wholly or partially covered by the user's hands or fingers as they hold the e-cigarette to inhale through it.

Embodiments of the present disclosure propose positioning the air inlet to address this issue. In particular, the air inlet can be positioned so that air reaching it is not drawn directly in through a wall of aerosol delivery section 14, but instead is caused to flow over a part of the battery section 12 before reaching the air inlet. This can be achieved by locating the air inlet in an interface region where part of the aerosol delivery section faces part of the battery section, and shaping and configuring parts of the battery section and/or the aerosol delivery section in this interface region to facilitate this air flow by providing air flow communication between the air inlet and an external opening to air formed at the edge of the interface region where the aerosol delivery section is adjacent to the battery section. Arrangements in accordance with this proposal can configure this external open end of the air flow path so that the risk of the path being blocked when holding the device is reduced or avoided.

FIGS. 2A and 2B are highly schematic representations of an e-cigarette 10 having a battery section 12 and an aerosol delivery section 14, to illustrate example positions of the interface region. The sections may be differently shaped than shown, with a differently shaped interface; these representations are examples only. The interface region is considered to be any area in which an outer surface or wall of the aerosol delivery section 14 faces an outer surface or wall of the battery section 12 when the two sections are joined together. In the side view of FIG. 2A, an interface region of interest is shown by the heavy line I, and comprises the area in which the base part of the aerosol delivery section 14 is received on the upper face of the connecting portion 28b, and the area in which a side wall of the aerosol delivery section 14 faces an adjacent side wall of the battery section 12. Note that the interface region extends through the thickness of the e-cigarette over the whole extent of the facing surfaces, and is not limited to the edges only. FIG. 2B shows a plan view of the system 10 from above, from which it can be seen that the interface region I between the side walls of the sections 12, 14 extends up to the top surface of the e-cigarette 10. In accordance with embodiments of the disclosure, the air inlet for the airflow channel is located on a surface of the aerosol delivery section 14 that is within the interface region, and at least one external opening to air that delivers air to the air inlet is located at an edge of the interface region, such as is indicated by the lines I in FIGS. 2A and 2B.

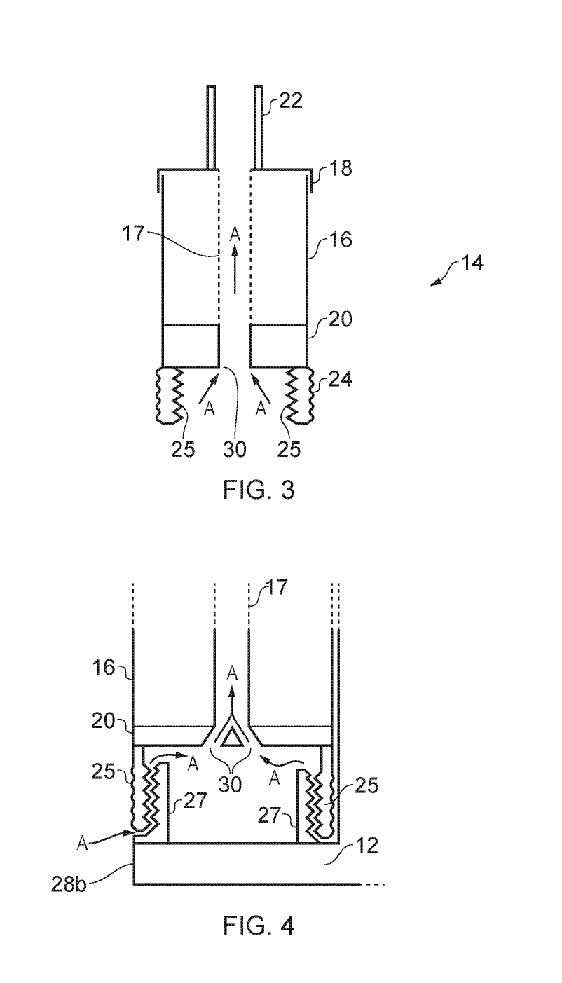

FIG. 3 shows a cross-sectional schematic view of an aerosol delivery section 14 configured according to some embodiments. In this example, the grip section 24 has a screw thread 25 on its inner surface for coupling with a corresponding thread on the battery section 12. Above this lies the tank base section 20, which in this example forms a base wall of the aerosol delivery section 14. Other configurations may be envisaged where parts other than the tank base form the base wall, for example if other components are installed below the tank 16, or if a connecting mechanism other than a screw thread is employed, such as a push or snap fit. The base wall faces the upper face of the connecting portion 28b of a battery section when a battery section is joined to the aerosol delivery section, giving an interface region. Regardless of the nature of the base wall, in this embodiment the air inlet 30 is formed in this base wall, and connects with the pipe 17 so that air taken in through the inlet 30 flows along the flow path and into the pipe 17 to the heating coil or element (not shown) and subsequently out through the mouth piece 22, as indicated by the arrows A. The air inlet 30 need not be centrally located as illustrated, and may also comprise two or more separate inlets which each connect with the pipe 17. The term “air inlet” is intended to cover arrangements with a single inlet and also with more than one inlet where these inlets each supply air to the airflow path.

To enable air intake into the air inlet 30 when the aerosol delivery section 14 is joined to its battery section, embodiments of the disclosure propose various configurations for one or more external aperture or openings to air displaced from the air inlet 30 and located at the interface edge, but in airflow communication with the air inlet 30. This allows the initial air entry point for the airflow path to be situated so that the risk of accidental blockage is mitigated. In some examples, this is achieved by moving this initial intake to a relatively low position on the e-cigarette so that the risk of a user blocking the air flow with his hand is reduced.

FIG. 4 shows a cross-sectional schematic view of a vapor provision system in accordance with such an embodiment. The aerosol delivery section 14 is largely as in FIG. 3, except that the air inlet 30 in this example comprises a plurality of individual inlets in an annular arrangement that connect by channels to the pipe 17 (or the air inlet might be a single annular aperture). The aerosol delivery section 14 is shown coupled to the battery section 12 via the screw thread 25 which is engaged with a corresponding screw thread 27 upstanding from the upper face of the connecting portion 28b of the battery section 12. Note that for simplicity the electrical connection is not shown. The screw threads are configured such that when they are fully tightened together there are one or more gaps extending through the threads that form an air flow pathway or channel by which air can enter from the external environment, traverse the screw thread into the volume of the interface region (under the base wall 20 of the tank 16) and so through the air inlet 30 to the pipe 17, as shown by the arrows A. This might be achieved by having breaks in the screw thread, or otherwise forming the screw threads so that when coupled they form a seal which is leaky or not airtight. By using the screw thread in this way, the external opening to air may be caused to extend around a large part of the lower edge (circumference) of the aerosol delivery section 14 so that it is very unlikely that a user would completely cover the air intake in use.

Alternative engagement arrangements between the aerosol delivery section 14 and the battery section can be configured to enable air intake in a similar manner to a leaky screw thread. For example, the aerosol delivery section and the battery section may be provided with cooperating engaging elements that are shaped to provide a friction fit when the two sections are pushed together or to provide a mechanical attachment by means of protruding lugs, a collar or similar element on one section that fit over or into depressions or hollows in the other section when the two sections are pushed together. These elements can be shaped so that when the sections are coupled or joined the resulting attachment is not airtight, and allows air to pass through to reach the air inlet in the base of the aerosol delivery section.

Other arrangements are possible in which the air inlet is located in the base wall of the aerosol delivery section, the interface region being between the aerosol delivery section base and the upper surface of the connecting portion.

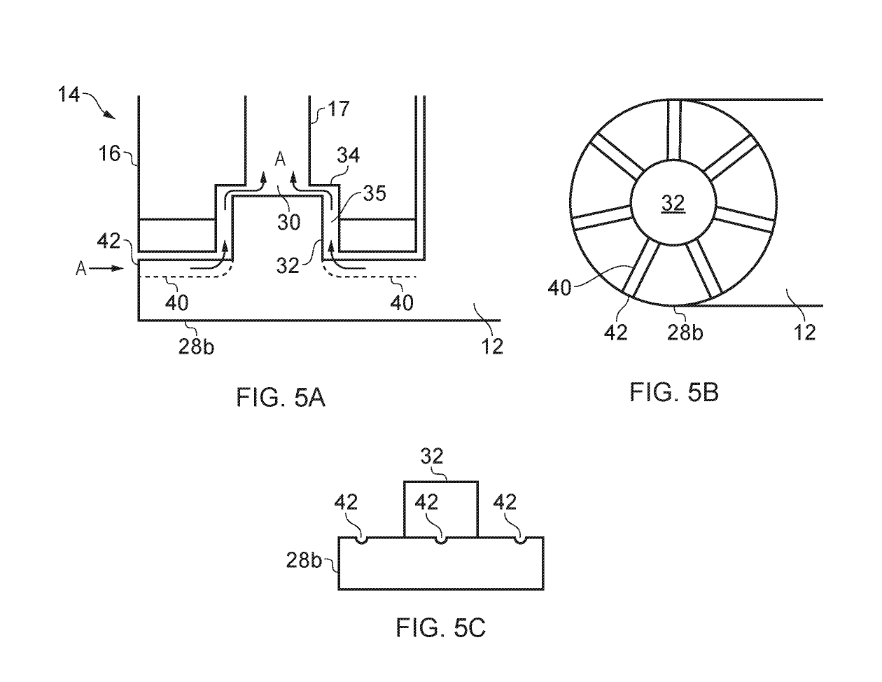

FIG. 5A shows a cross-sectional view through the interface region of an embodiment having a base wall air inlet. The aerosol delivery section 14 is joined to the connecting portion 28b of the battery section 16. It has a socket 34 which engages with a connector 32 on the connecting portion 28b (as in FIG. 1A) to make the required electrical connection (not shown). A central air inlet 30 is formed in the base wall, within the socket 34. The socket is shaped so as to be larger than the connector so that there is a cavity 35 between the walls of the socket 34 and the walls of the connector 32, which is in airflow communication with the air inlet 30. To enable air to enter this cavity and thence the air inlet 30, the upper face of the connecting portion 28b of the battery section 16 has formed in it a number of recesses or grooves 40. The grooves have one end at or near to the connector 32 so that this end opens into the cavity between the connector 32 and the socket 34. In this example, the aerosol delivery section 14 has the same outer profile as the connecting section 28b at the interface region edge, so the grooves 40 have opposite ends in the side walls of the connecting section 28b, to form an external opening to air 42 at the end of each groove 40. Thus, when the aerosol delivery section is connected, air can enter the external openings, flow along the grooves 40 (under the base wall of the aerosol delivery section 14 and over those parts of the surface of the connecting portion that form the grooves) to the cavity and into the air inlet 30.

FIG. 5B shows a plan view (to a different scale) of the connecting portion 28b, extending laterally from the battery section 16. In this example, there are seven grooves 40 arranged radially so that they extend inwardly from the ends in the side walls of the connecting portion towards the central connector 32. In effect, they are radially arranged with respect to a central longitudinal axis of the aerosol delivery section 14 when it is joined to the battery section 16. However, other arrangements are possible, for example if the connector and socket are not centrally disposed, or if a different configuration is otherwise preferred. For example, there may be a plurality of parallel grooves arranged with one end at the outer edge and one end reaching to the cavity. Groups of parallel grooves might be spaced apart around the connecting portion. Any number of grooves, from one upwards, can be used as required to achieve a desired level of air intake and airflow in the airflow path. The grooves need not be straight, and they may have a width that changes along their length. However, a larger number of grooves gives a larger number of external openings to air, which decreases the risk of the air intake being blocked by a user holding the e-cigarette. The grooves together can be thought of as comprising part of the cavity 35, so that the outer ends 42 of the grooves or recesses are the external openings to air of the cavity.

FIG. 5C shows a side view (to a different scale) of the connecting portion 28b, depicting how the grooves extend out to the side walls of the connecting portion 28b so that their ends 42 appear as notches.

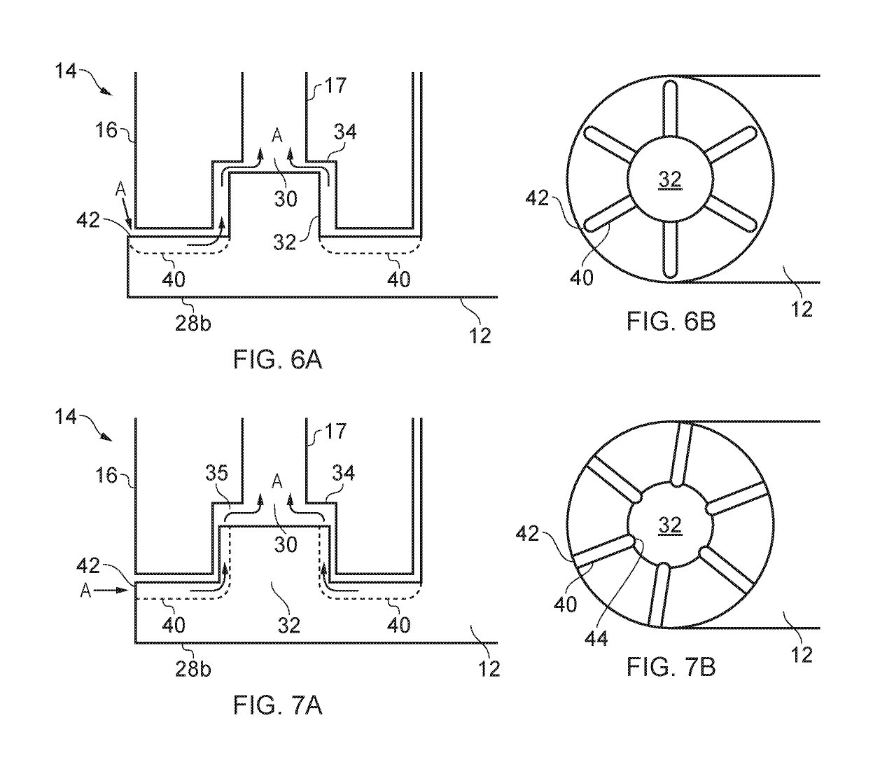

FIG. 6A shows an alternative embodiment comprising grooves or recesses in the connecting portion upper face. Many features are the same as in the example of FIGS. 5A to 5C, and the remarks made regarding that example apply also to this example. A difference from the FIG. 5A example is that the aerosol delivery section 14 has a smaller width (diameter in this cylindrical example) than the connecting portion 28b on which it sits, so that the edges of the connecting portion 28b extend beyond the base of the aerosol delivery section 14. This means that there is no need for the grooves to extend out into the side walls of the connecting portion 28b. Instead, they terminate in the upper face of the connecting portion 28b at a point beyond the outer edge of the aerosol delivery section, to form the external openings to air 42.

FIG. 6B shows a corresponding plan view (to a different scale) of the connecting portion 28b, in which it can be seen that the grooves (six in this example) are again radially arranged, but have outer ends located inwardly from the edge (side wall) of the connecting portion 16.

FIG. 7A shows a further alternative embodiment comprising grooves or recesses in the connecting portion upper face. Again, many features are the same as in the example of FIGS. 5A to 5C, and the remarks made regarding that example apply also to this example. A difference from the FIG. 5A example is that the socket 34 and the connector 32 have closely matched diameters (or other width dimensions if not circular) so that the cavity 35 is limited to only a space above the connector 32 and below the air inlet 30 in the base of the socket 34. To enable air to reach the cavity 35 to enter the air inlet 30, the grooves 40, again in the upper face of the connecting portion, extend also up the sides of the connector 32 and terminate in the upper face of the connector 32 to communicate with the cavity 35. The outer ends 42 of the grooves 40 defining the external openings to air are formed in the outer wall of the connecting portion 28b, as in FIG. 5A, but may instead be formed as in FIG. 6A.

FIG. 7B shows a corresponding plan view (to a different scale) of the connecting portion 28b, in which it can be seen that the grooves (five in this example) are again radially arranged, have outer ends 42 in the side wall of the connecting portion 16, and inner ends 44 in the upper face of the connector 32 to give airflow communication into the cavity 35.

In other embodiments, the air inlet can be located in the side wall of the aerosol delivery section, so as to take in air received via the interface region between the side wall of the aerosol delivery section and the facing side wall of the battery section. This arrangement moves the air inlet away from electrical connection. This can be beneficial in reducing the risk of any leakage of the source liquid from the tank to the electrical contacts via the airflow pathway.

To achieve such an arrangement, the outer surface of the battery section can be shaped so as to include one or more recesses or grooves in the wall that faces and abuts the aerosol delivery section. The recesses extend out to the edge of the interface region. When the sections are placed together, this shaping defines one or more cavities (multiple cavities are considered still as one cavity for understanding of these embodiments) in the interface region, which have an external communication to air via ends of the recesses that reach to the edge of the interface region and form openings thereat. The air inlet (which might be one or more individual apertures that connect with the pipe 17 as described with regard to FIGS. 3 and 4) is created in the aerosol delivery section in a position that is aligned with the cavity. Thus, air can be taken into the airflow path by entering through the external opening(s) into the cavity and then into the air inlet.

The aerosol delivery section and the battery section can be configured to ensure that the alignment of the air inlet with the cavity is achieved and maintained when the two sections are joined. For example, there may be shaped co-operating parts that allow the sections to be engaged only in the required orientation, or a screw thread coupling may be structured to that the air inlet and the cavity are brought into alignment when the screw thread is fully fastened.

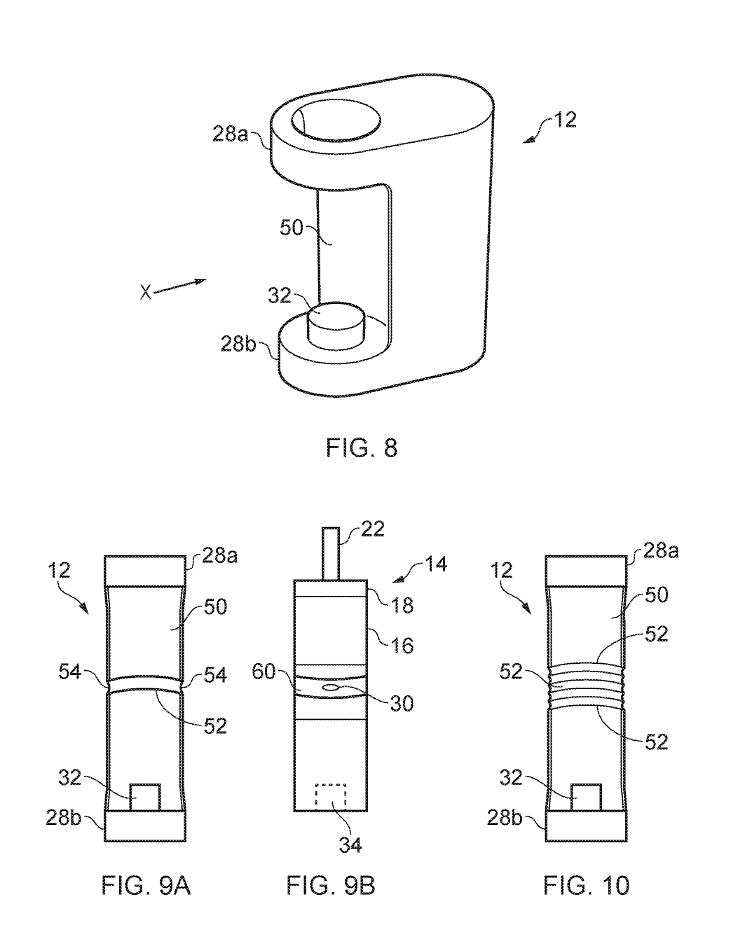

FIG. 8 shows a perspective view of an example battery section 12 such as that shown in FIG. 1. Supporting sections 28a, 28b including connecting portion 28b extend laterally from the battery section to receive, support and hold an aerosol delivery section, and make an electrical connection to the aerosol delivery section via a connector 32. The battery section 12 has a side wall 50 which will face a side wall of a joined aerosol delivery section. The interface region lies in the area of these side walls in the following embodiments.

FIG. 9A shows a side view of a battery section 12 from the direction of arrow X in FIG. 8. In this example, the side wall 50 has a groove 52 defined in its surface, extending substantially horizontally across the wall 50 from one side to the other. The groove 52 terminates at the edges of the wall, in this example in the form of notches 54 in the edges. Hence, the groove ends 54, which form the external openings to air in this embodiment, will be externally visible in this example. Alternatively, certain shapes of battery section, particularly those like this example where the side wall 50 is concave, will allow the groove ends 54 to be formed so as to be less visible.

FIG. 9B shows a schematic side view of an aerosol delivery section 14 suitable for use with the battery section 12 of FIG. 9A. An air inlet 30 is formed in the side wall 60. In this example, the air inlet 30 is near the base of the tank 16, but it may be disposed elsewhere in the interface region, with corresponding positioning of the groove 54 so that the air inlet 30 and the groove can be aligned. In use, the two sections are joined so that the side wall 60 of the aerosol delivery section faces the side wall 50 of the battery section 12, defining the interface region.

The groove 52, when brought together with the side wall 60 of the aerosol delivery section 14, can be thought of as a cavity of a particular shape, having two external openings to air (one each side of the battery section). A groove or recess with only one external end might be provided instead, but the use of more than one external opening to air reduces the risk of accidental blockage of the air intake. Additionally, cavities of other shapes and configurations, with various numbers of external openings, may be used instead.

FIG. 10 shows a second example of a battery section 12, in side view, configured with a shaped side wall. In this example, three substantially parallel horizontal grooves are provided, each extending to both edges of the side wall 50 to give a total of six external openings to air. To function with this cavity arrangement, the aerosol delivery section might have three individual air inlets each connected to the airflow path, one for each groove 52, or a single air inlet having a large vertical extent that traverses all three grooves 52.

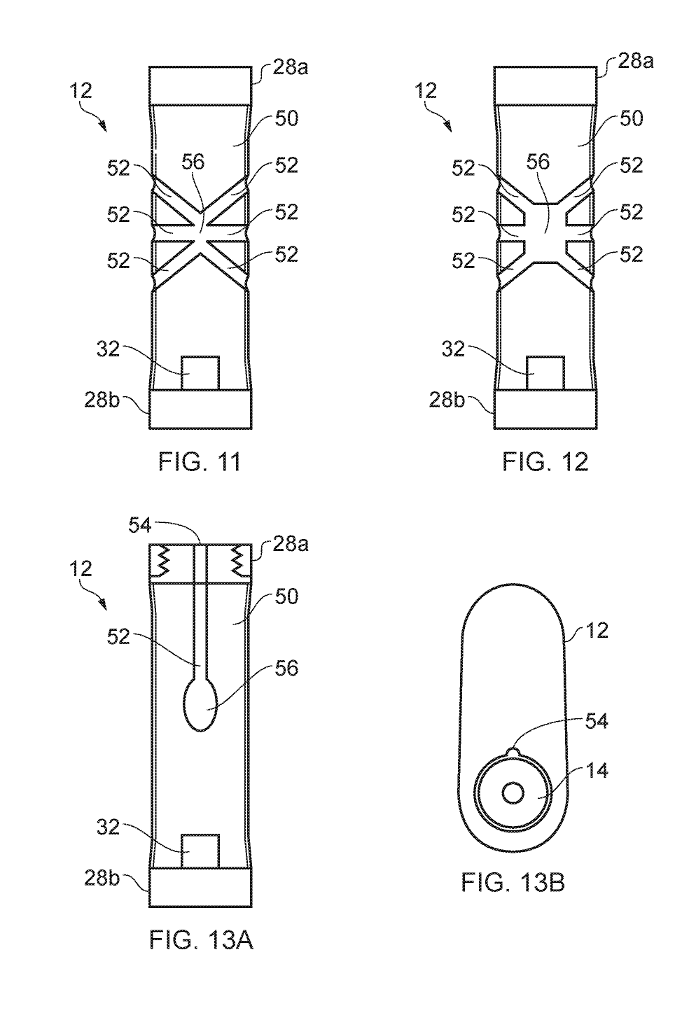

FIG. 11 shows another example of a battery section 12, in side view, configured with a shaped side wall. In this example, a total of six grooves 52 are provided to define with cavity, three of which create external openings to air on one edge of the side wall 50 and three of which create external openings to air on the opposite side of the side wall 50. The grooves are angled so as to all meet together at their ends opposite to the external opening ends, forming a star shape. An arrangement such as this, in which multiple external openings connect to a single central recess 56 to which the air inlet will be aligned, provide the advantage of multiple air intake positions while avoiding any need for a large air inlet or multiple air inlets.

FIG. 12 shows a similar example to that of FIG. 11. In this example, again shown in side view, the six grooves 52 in the side wall 50 meet at a much larger central recess 56. This provides the advantages of the FIG. 11 example together with more flexibility in the positioning of the air inlet for proper alignment with the cavity. This can give improved design freedom, for example, or require a less precise fit or coupling when a user joins the aerosol delivery section to the battery section 12.

Embodiments of this type in which recesses are formed in the battery section side wall are not limited to configurations in which the recesses define external openings to air at the sides of the e-cigarette. The recesses or grooves may alternatively or additionally be arranged to as to define one or more external openings at the top of the device. To achieve this, the recess(es) may be aligned more vertically.

FIG. 13A shows a side view of an example battery section 12 configured in this way. The upper supporting portion 28a is shown cut away to reveal the top part of the side wall 50. A vertical groove 52 is shaped into the side wall 50 extending from the top edge where it defines an external opening to air 54, down to a larger recess 56 where the air inlet on the aerosol delivery section can be aligned. FIG. 13B shows a plan view of the top of the battery section 12, showing how the groove forms the external opening to air 54 at the top edge of the wall when the aerosol delivery system 14 is inserted into the battery section 12.

More than one vertical groove might be provided, which may or may not terminate in a common recess. Also, one or more substantially vertical grooves may be combined with one or more substantially horizontal grooves, so provide external openings to air at both the top and sides of the device. The sides of the device offer more space to accommodate a larger number of air intakes, but since these are more vulnerable to being covered in use than an intake at the top of the device, a top intake can be provided as well to ensure that there is at least one intake that is highly unlikely to be accidentally blocked.

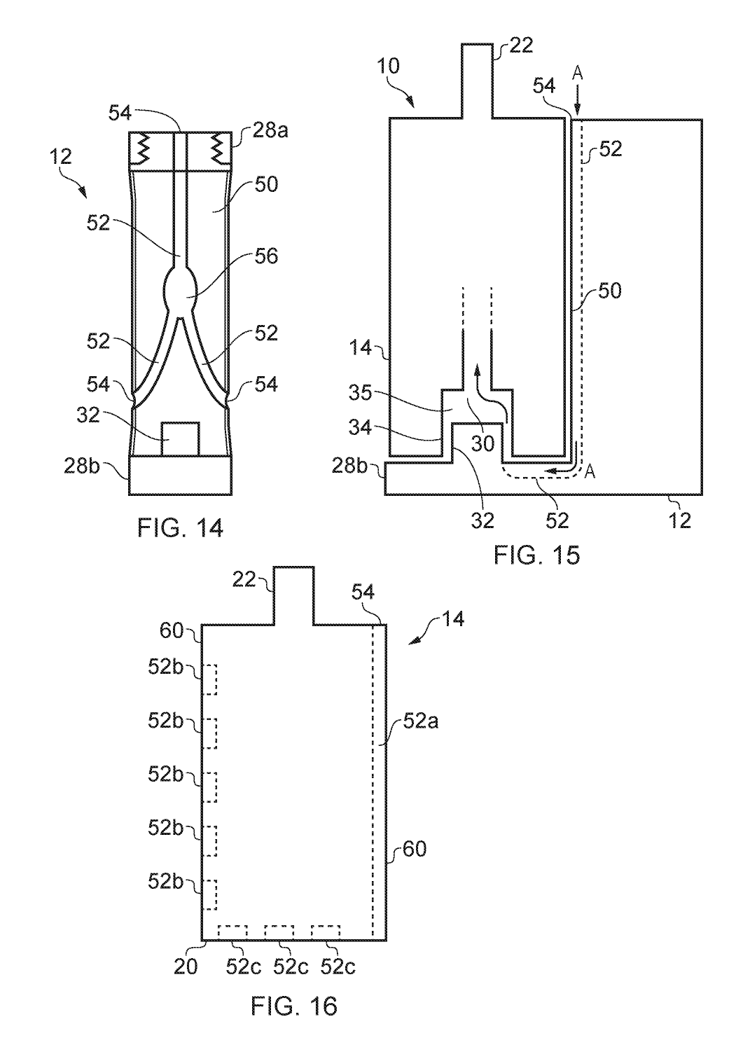

FIG. 14 shows a side view of an example battery section configured in this manner. It has the same features as the battery section shown in FIG. 13A, but also includes two additional grooves 52 which extend between external openings to air 54 in opposite side edges of the interface region and the larger recess 56. More such grooves or just one such groove might be included if desired.

A further alternative is to combine a groove or recess in the battery section side wall 50 with one or more grooves or recesses in the connecting portion such as in the examples of FIGS. 5, 6 and 7. A groove in the battery section side wall may commence at an external opening in an edge of the interface region, and extend to the lower part of the battery section side wall to connect to a groove in the connection portion upper surface, to provide an air pathway to an air inlet in the base wall of the aerosol delivery section.

FIG. 15 shows a schematic cross-sectional view of an example vapor provision device 10 having these features. The battery section 12 has formed in its side wall 50 a vertical groove or recess 52 extending from an external opening to air 54 at its top edge adjacent the mouthpiece 22 of the aerosol delivery section 14 down to the bottom edge of the side wall 50, where it connects to a groove 52 in the upper face of the connecting portion 28b. This groove 52 is in airflow communication with a cavity 35 between the connector 32 of the connecting portion 28b and the socket 34 in the base of the aerosol delivery section 14. In this way, air taken in at the external opening 54 at the top of the system flows along the connected grooves 54 to the cavity 35 and into the air inlet 30, to enter the airflow path through the aerosol delivery section 14.

Grooves and recesses of any size, shape, position and quantity can be provided to carry air from the edge of the interface region (the exterior junction between the aerosol delivery section and the battery section) to an air inlet located inside the interface region. The grooves need not be straight or of constant width, and may meet together or remain separate. Each groove, or the overall cavity defined by one or more grooves or recesses, may have one or more than one external opening to air.

The examples and embodiments discussed above have utilized various shapings in the outer surface of the battery section to achieve the desired channeling of air from the external intake(s) to the interior air inlet on the aerosol delivery section. Equivalent effects can be readily achieved by using shaping of the outer surface of the aerosol delivery section instead, or combining shaping of the aerosol delivery section and the battery section. Consequently, all such combinations are considered to be within the scope of the claimed invention. Moreover, precise shapings such as those described herein may not be required. Instead the aerosol delivery section and the battery section may be configured such that when they are joined, the facing surfaces are spaced apart sufficiently to define an air gap in communication with the external air over part or all of the interface region.

FIG. 16 shows a highly schematic and simplified side view of an aerosol delivery section with shapings in its outer surface to channel air into an air inlet in an interface region. Note that the air inlet or inlets are not shown in this illustration, but may be positioned where desired to be in airflow communication with air guided by the shapings. Three example positions for possible shapings are shown in phantom; an individual device may have one or some of these only. As a first example, the aerosol delivery section 14 has a vertical groove 52a in a side wall 60 which in use will face a battery section, with an external opening to air 54 at its upper end. This is functionally equivalent to the vertical groove 52 in the battery section shown in FIG. 15. One or more vertical grooves or recesses 54a might be formed, which may or may not extend the full height of the aerosol delivery section 14. As a second example, the aerosol delivery section 14 has a series of horizontal grooves 52b formed in a side wall 60 which in use will face a battery section. Any number of such grooves (which may have any shape, such as a corrugation or concertina shape if many grooves are desired) may be included, which may come together to feed a single air intake, or may each feed a separate air intake. A third example is a series of grooves or recesses 54c in the base wall 20 of the aerosol delivery section 54, which will face the upper surface of a connecting portion of a battery section when the two sections are joined. These grooves 54c are in airflow communication with an air inlet in the base wall 20, such as in FIGS. 3, 5A, 6A and 7A, such as via a cavity 35 as in FIG. 5A, 6A or 7A, and might be radially arranged similar to the grooves in the FIG. 5B example.

Any of the embodiments and examples presented herein may further comprise an airflow adjuster by which a user can modify the level of airflow in the airflow path and hence control the amount of aerosol delivered per inhalation. Any suitable adjuster may be employed. For example, the adjuster may comprise a movable element such as a curved or flat plate or ring which is slidable over the air inlet so as to partially cover the air inlet and alter an area of the effective bore of the air inlet. The adjuster is preferably configured such that the air inlet cannot be completely covered by the movable element (which would block the airflow path), and may be configured for continuous adjustment or for stepped adjustment between two or more predetermined air inlet sizes and the corresponding air flow levels.

The various embodiments described herein are presented only to assist in understanding and teaching the claimed features. These embodiments are provided as a representative sample of embodiments only, and are not exhaustive and/or exclusive. It is to be understood that advantages, embodiments, examples, functions, features, structures, and/or other aspects described herein are not to be considered limitations on the scope of the invention as defined by the claims or limitations on equivalents to the claims, and that other embodiments may be utilized and modifications may be made without departing from the scope of the claimed invention. Various embodiments of the invention may suitably comprise, consist of, or consist essentially of, appropriate combinations of the disclosed elements, components, features, parts, steps, means, etc., other than those specifically described herein. In addition, this disclosure may include other inventions not presently claimed, but which may be claimed in future.

1. A vapor provision system comprising:
an aerosol delivery section configured to generate aerosol from liquid in a reservoir;
an airflow path through the aerosol delivery section extending from an air inlet to a mouthpiece;
a battery section configured to join to the aerosol delivery section and house a battery to provide electrical power to one or more components in the aerosol delivery section, the battery section arranged laterally to at least a portion of the aerosol delivery section with respect to a direction of airflow through the mouthpiece; and
an interface region in which a surface of the aerosol delivery section faces a surface of the battery section when the aerosol delivery section and the battery section are joined;
wherein the air inlet is located on the aerosol delivery section in the interface region so as to take in air that has been channeled over part of the battery section.

2. The vapor provision system according to claim 1, wherein the battery section is arranged laterally to the aerosol delivery section with respect to a direction of airflow through the mouthpiece.

3. The vapor provision system according to claim 1, wherein the battery section has a connecting portion that extends laterally to receive a base part of the aerosol delivery section, and the air inlet is located in a base wall of the aerosol delivery section that faces the connecting portion when the battery section and the aerosol delivery section are joined.

4. The vapor provision system according to claim 3, comprising co-operating screw threads or engaging elements on the connecting portion and the aerosol delivery section base part by which the battery section and the aerosol delivery section can be joined, the screw threads or engaging elements being shaped such that when the screw threads or engaging elements are fully engaged, air can flow over at least part of the screw threads or engaging elements to be taken in by the air inlet.

5. The vapor provision system according to claim 3, wherein a surface of the connecting portion that faces the base part of the aerosol delivery section has formed therein at least one recess such that when the battery section and the aerosol delivery section are joined a cavity is formed in the interface region with at least one external opening to air at an edge of the interface region, the air inlet being in airflow communication with the cavity.

6. The vapor provision system according to claim 5, wherein the at least one recess comprises at least one groove in the surface of the connecting portion, the at least one groove extending radially with respect to a central axis of the aerosol delivery section when joined to the battery section to an end that forms one of the at least one external openings.

7. The vapor provision system according to claim 6, wherein the base part of the aerosol provision section and the surface of the connecting portion are shaped to form a central cavity in the interface region with which the at least one groove is in airflow communication at an end opposite to the external opening end, the air inlet being in airflow communication with the central cavity.

8. The vapor provision system according to claim 7, wherein the central cavity houses an electrical connection between the battery section and the aerosol delivery section.

9. The vapor provision system according to claim 1, wherein the aerosol delivery section and the battery section are externally shaped such that when the aerosol delivery section and the battery section are joined a cavity is formed in the interface region with at least one external opening to air at an edge of the interface region, the air inlet being in airflow communication with the cavity.

10. The vapor provision system according to claim 9, wherein the surface of the battery section in the interface region is a side wall of the battery section and the surface of the aerosol delivery section in the interface region is a side wall of the aerosol delivery section.

11. The vapor provision system according to claim 9, wherein the cavity is at least partially formed by at least one recess in the side wall of the battery section having at least one end that forms one of the at least one external openings to air.

12. The vapor provision system according to claim 11, wherein the at least one recess is a groove extending across the side wall of the battery section from a first end to a second end, each of the first end and the second end forming one of the at least one external openings to air.

13. The vapor provision system according to claim 11, wherein the at least one recess has an end forming one of the at least one external openings to air, the end located at an edge of the interface proximate the mouthpiece.

14. The vapor provision system according to claim 5, wherein the cavity has at least two external openings to air.

15. The vapor provision system according to claim 5, further comprising an adjustable element configured to enable an effective size of the air inlet to be altered, so as to vary a level of airflow along the airflow path.

16. An aerosol delivery section for a vapor provision system which is configured to generate aerosol from liquid in a reservoir when joined to a battery section housing a battery, the aerosol delivery section comprising:
an air inlet;
an airflow path through the aerosol delivery section from the air inlet to a mouthpiece; and
an interface region comprising a surface of the aerosol delivery section configured to face a surface of a battery section when the aerosol delivery section is joined to the battery section;
wherein the air inlet is located in the interface region so as to take in air that has been channeled over part of the battery section when the aerosol delivery section and the battery section are joined.

17. A battery section for a vapor provision system which is configured to house a battery to provide electrical power to an aerosol delivery section when the battery section is joined to an aerosol delivery section, the battery section comprising:
an interface region comprising a surface of the battery section configured to face a surface of an aerosol delivery section when the battery section is joined to the aerosol delivery section; and
at least one recess formed in the interface region positioned for alignment with an air inlet in the aerosol delivery section, the at least one recess having at least one end at an edge of the interface region to define an external opening to air.
